(19) United States
(12) Patent Application Publication
(10) Pub. No.: US 2008/0053445 A1
(43) Pub. Date: Mar. 6, 2008

(54) Cardiopulminary resuscitation timer

(76) Inventors: Kevin D. Kroupa, Ballwin, MO (US); James A. Wilson, Fairview Heights, IL (US); Christopher A. Scholz, Fenton, MO (US)

Correspondence Address: GREENSFELDER HEMKER & GALE PC, SUITE 2000, 10 SOUTH BROADWAY, ST LOUIS, MO, 63102, US

(21) Appl. No.: 11/511,674

(22) Filed: Aug. 29, 2006

(51) Int. Cl. A61M 16/00; A62B 9/00

(52) U.S. Cl. 128/205.23; 128/204.23

(57) ABSTRACT

A cardiopulmonary resuscitation (CPR) timer for use with a bag mask resuscitator provides a visual pacing device when a cardiopulmonary resuscitation procedure is being performed on a patient is disclosed. The CPR timer includes a plurality of visual indicators arranged in an array that sequentially activate at a predetermined pace that matches the recommended pace for air delivery times and/or chest compressions when using a bag mask resuscitator to provide life support to a patient. The CPR timer further includes a pressure sensor in operative association with the bag mask resuscitator for detecting when the bag mask resuscitator is actuated.

### FIELD

The present document relates to a bag mask resuscitator, and more particularly to a cardiopulmonary resuscitation (“CPR”) timer used with the bag mask resuscitator.

### SUMMARY

In an embodiment a cardiopulmonary resuscitation timer may include a housing, said housing including a plurality of visual indicators arranged in an sequence, said plurality of visual indicators being adapted to be sequentially activated at a predetermined pace as a visual pace setting device for pacing breaths and/or chest compressions to a patient.

In another embodiment, a bag mask resuscitator may include a hollow, flexible resuscitation bag in fluid flow communication with a valve assembly through a hollow, flexible hose, said valve assembly being in operative engagement with a mask, said valve assembly including an adaptor defining an outlet port adapted to engage a hollow flexible tubing, and a cardiopulmonary resuscitation timer defining an inlet port engaged to said tubing, said tubing in operative association with a pressure sensor for monitoring air pressure being applied by the bag mask resuscitator through the tubing, said cardiopulmonary resuscitation timer further including a plurality of visual indicators for providing a predetermined pace for actuating said resuscitation bag.

In yet another embodiment, the bag mask resuscitator may include a hollow, flexible resuscitation bag in fluid flow communication with a valve assembly, said valve assembly being in operative engagement with a mask, said valve assembly including an adaptor defining an outlet port adapted to engage a hollow flexible tubing, and a cardiopulmonary resuscitation timer defining an inlet port engaged to said tubing, said tubing in operative association with a pressure sensor for monitoring air pressure being applied by the bag mask resuscitator through the tubing, said cardiopulmonary resuscitation timer further including a plurality of visual indicators for providing a predetermined pace for actuating said resuscitation bag.

Implementation of the above embodiments may include one or more of the following features:

The plurality of visual indicator includes a start indicator and a plurality of timing indicators.

The activation of said start indicator provides a visual cue to the user to begin providing breaths and/or chest compressions to a patient.

The plurality of timing indicators are sequentially activated in order to provide a visual pace setting device for pacing the frequency of breaths and/or chest compressions provided to a patient after the start indicator has been activated.

Each of said plurality of visual indicators is an LED.

The predetermined pace includes providing a pace established for either an adult or a child.

The visual indicators are deactivated in reverse sequence during an expiratory time period.

The cardiopulmonary resuscitation timer further includes a pressure sensor for sensing air pressure indicative of a breath being provided to a patient.

The cardiopulmonary resuscitation timer further including a pressure sensor for sensing air pressure indicative of a breath being provided to a patient, wherein said start indicator is activated when the air pressure sensed by said pressure indicator exceeds a predetermined threshold.

The plurality timing indicators are one color and said start indicator is a different color.

The cardiopulmonary resuscitation timer further includes an inspiratory time indicator for providing the amount of inspiratory time taken to provide one or more breaths to a patient.

The cardiopulmonary resuscitation timer further includes a breaths per minute indicator for indicating the number of breaths per minute provided to a patient.

The cardiopulmonary resuscitation timer further includes airway pressure indicator in operative association with said pressure indicator for indicating the presence of air pressure.

The timing indicators may be non-visual indicators.

The cardiopulmonary resuscitation timer is operable between a CPR operation mode and a rescue breathing operation mode.

The cardiopulmonary resuscitation timer further includes a mode change selector for changing the mode of operation between said CPR operation mode and a rescue breathing operation mode.

The cardiopulmonary resuscitation timer further includes a patient selector for providing different kinds of said predetermined pace when operating in either said CPR operation mode or a rescue breathing operation mode.

Additional objectives, advantages and novel features will be set forth in the description which follows or will become apparent to those skilled in the art upon examination of the drawings and detailed description which follows.

### BRIEF DESCRIPTION OF THE DRAWINGS

FIG. 1 is a side view of a bag mask resuscitator with a CPR timer;

FIG. 2 is a front plan view of the CPR timer;

FIG. 2A is a front plan view illustrating the connection of a flexible tubing with a pressure sensor inside the CPR timer;

FIG. 3 is a schematic circuit diagram of the CPR timer;

FIG. 4 is a flowchart illustrating a method of operating the CPR timer;

FIG. 5 is a flowchart illustrating a method of sampling a power source;

FIG. 6 is a flowchart illustrating a method of sampling a sensor; and

FIG. 7 is a timing diagram of a breath using the bag mask resuscitator.

Corresponding reference characters indicate corresponding elements among the several views. The headings used in the figures should not be interpreted to limit the scope of the figures.

### DETAILED DESCRIPTION

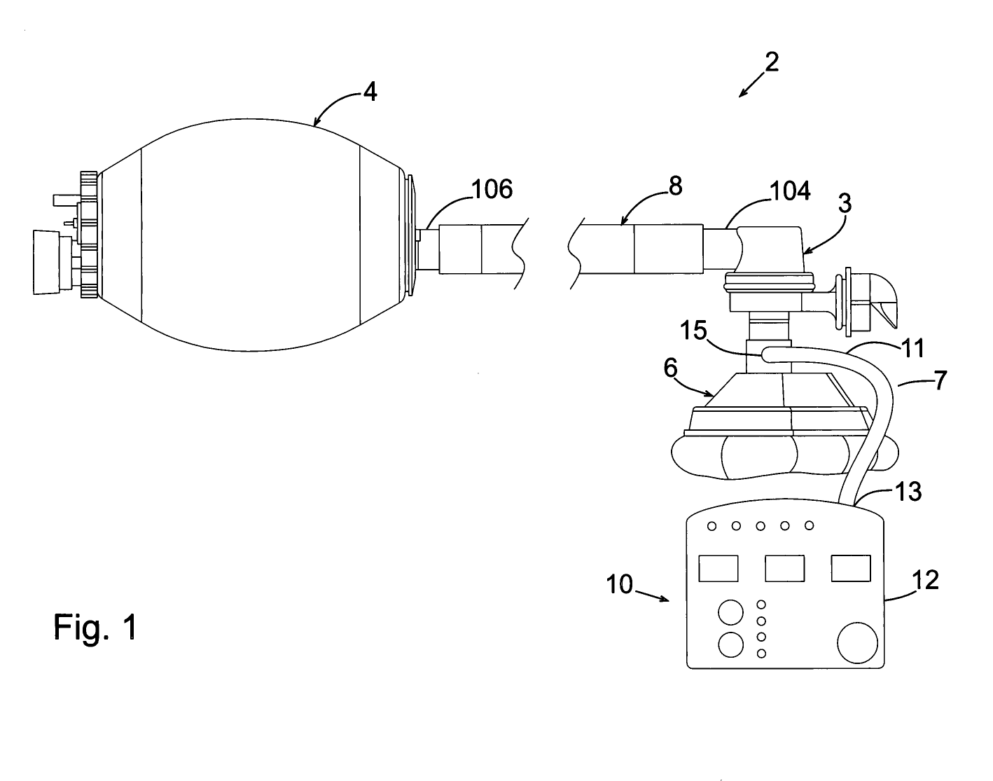

Referring to the drawings a cardiopulmonary (“CPR”) timer for use with a bag mask resuscitator 2 is illustrated and generally indicated as 10 in FIG. 1. In one embodiment, CPR timer 10 may be used with bag mask resuscitator 2 for providing a visual pace setting device to pace the user's actuation of the bag mask resuscitator 2 and/or pace the number of chest compressions provided to a patient during emergency life saving support. Bag mask resuscitator 2 may include a hollow, flexible resuscitation bag 4 that provides oxygen to the patient when the user repeatedly squeezes the resuscitation bag 4 to force oxygen through a flexible, hollow hose 8 and into the patient's respiratory system through a mask 6 seated on the patient's face which is in fluid flow communication with the resuscitation bag 4. Bag mask resuscitator 2 may also provide air to the patent as an option to providing oxygen as herein described.

Bag mask resuscitator 2 may also provide air to the patient as an option to providing oxygen as herein described.

Bag mask resuscitator 2 may provide emergency life saving support to a patient, for example when the patient suffers a heart attack and requires manual ventilation to force oxygen or air into the patient's respiratory system and/or chest compressions to the patient. As shown, the flexible, hollow hose 8 includes a distal end 104 attached to a valve assembly 3 and a proximal end 106 attached to the resuscitation bag 4 such that fluid flow communication is established between the valve assembly 3 and resuscitation bag 4.

The valve assembly 3 is operatively engaged to mask 6 which is seated on a patient's face for providing oxygen or air to the patient when the resuscitation bag 4 is actuated. As further shown, valve assembly 3 includes an adaptor 11 that defines an outlet port 15 adapted to be in fluid flow communication with the CPR timer 10 through a hollow, flexible tubing 7 for the passage of air flow therethrough caused by the actuation of resuscitation bag 4 in order to detect and determine current detected pressure by the CPR timer 10 as shall be discussed in greater detail below.

In an alternative embodiment, the resuscitation bag 4 may be directly engaged to the valve assembly 3 without use of the flexible hose 8. As noted above, the CPR timer 10 acts as a visual pace setting device to pace the user's actuation of the resuscitation bag 4 when the CPR timer 10 is in the rescue breathing operation mode so that the user can provide the recommended number of breaths per minute to the patient when providing emergency life saving support. For example, the CPR timer 10 may provide a visual pace setting that paces the user to provide a greater number of breaths to a child than to an adult when giving emergency life saving support.

Alternatively, in the CPR operation mode the CPR timer 10 acts as a visual pace setting device to pace the user's actuation of the resuscitation bag 4 in addition to pacing the number of chest compressions provided to a patient between each manual ventilation of a patient's respiratory system. For example, the CPR timer 10 may provide a visual pace setting that paces the user to provide a different number of chest compressions and breaths per minute to an adult than a child.

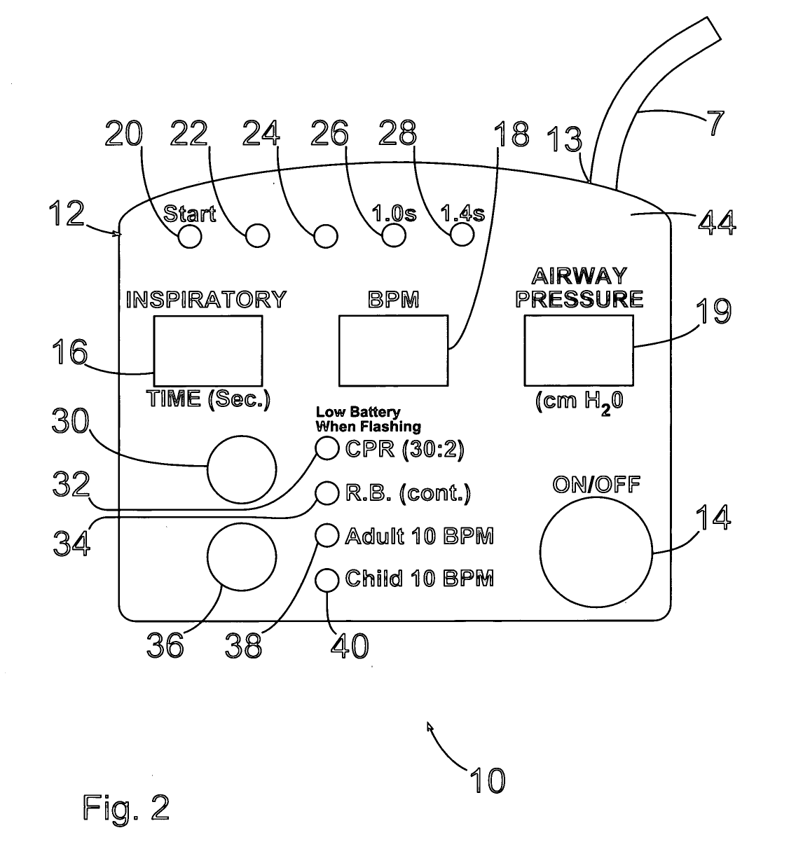

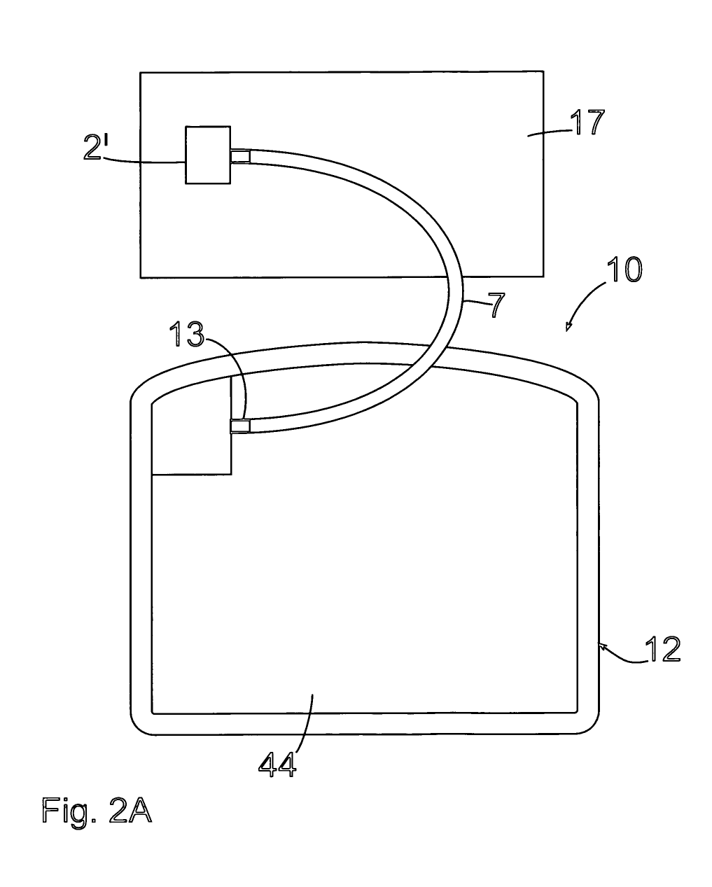

Referring to FIGS. 2 and 2A, CPR timer 10 may include a housing 12 having a circuit board 17 for providing the various pace setting operations required to visually pace the number of chest compressions applied to the patient or the user's actuation of the resuscitation bag 4 when manually ventilating the patient's respiratory system. As further shown, housing 12 defines an inlet port 13 adapted to engage tubing 7 for providing monitoring air pressure as shall be discussed in greater detail below. CPR timer 10 may further include an ON/OFF switch 14 for selectively permitting or preventing operation of the CPR timer 10. ON/OFF switch 14 may be an alternating action switch that permits power to flow to the electronics when the switch is closed. However, other types of switches, such as momentary switches and tactile switches, are also contemplated.

As further shown, housing 12 may define a front surface 44 which may be configured to have light diffusing characteristics. In one aspect, front surface 44 may be made from polycarbonate and define a textured finish. In addition, front surface 44 may have a dome or stepped shaped having multiple rectangles that decrease in size. However, front surface 44 may have other ergonomic shapes that provide a better gripping surface and enhanced viewing and use by the user. In one embodiment, housing 12 may be made from a molded plastic or metal, however other lightweight, durable and/or water resistant materials are contemplated.

CPR timer 10 may also include an inspiratory time indicator 16, Breaths Per Minute (BPM) indicator 18 and an airway pressure indicator 19 which are visible through front surface 44 to provide information to the user regarding various operations of CPR timer 10 as shall be discussed in greater detail below.

Inspiratory time indicator 16 may indicate the amount of inspiratory time being taken to provide one breath to the patient when actuating resuscitation bag 4. In one embodiment, inspiratory time indicator 16 may be two seven-segment light emitting diodes (LED display) for displaying inspiratory time, however other types of indicators such as liquid crystal displays (LCDs), are also contemplated. For example, the two seven-segment LEDs may be manufactured by Ledtech Electronics Corp.

BPM indicator 18 may indicate the number of breaths per minute being provided to the patient as the resuscitation bag 4 is being actuated by the user. In one embodiment, BPM indicator 18 may be two seven-segment LED display, however other types of indicators, such as LCDs, are also contemplated.

In addition, airway pressure indicator 19 may indicate the presence of air pressure above a predetermined threshold being detected through flexible 7. Airway pressure indicator 19 evidences actuation of the resuscitation bag 4 when the air pressure detected exceeds a predetermined air pressure threshold so that various pace setting operations of CPR timer 10 may be initiated.

As noted above, CPR timer 10 provides a means for visually pacing a user when operating the bag mask resuscitator 2. CPR timer 10 may include a start indicator 20 and timing indicators 22, 24, 26, 28 which may provide visual indications to the user for pacing breaths provided to the patient by actuation of resuscitation bag 4 as described in greater detail below.

The timing indicators 22, 24, 26 28 (with or without start indicator 20) may be sequentially illuminated in series at predetermined intervals to visually indicate the pace of breaths to be provided to the patient when actuating resuscitation bag 4. As such, the sequential illumination of timing indicators 22, 24, 26, 28 enables a user to visually follow timing indicators 22, 24, 26, 28 in order to pace the actuation of the resuscitation bag 4 and provide the proper number of breaths per minute to the patient.

In one embodiment, four timing indicators 22, 24, 26, 28 may be used with CPR timer 10 although other number of timing indicators are also contemplated for providing a visual pace setting indication. The timing indicators 22, 24, 26, 28 may be bi-color LEDs, however other visual and non-visual indicators for pacing the number of breaths to be provided to the client are also contemplated.

The operation of CPR timer 10 may be adjusted by actuating mode change selector 30 and patient selector 36 on housing 12. In operation, actuation of mode change selector 30 may enable a user of CPR timer 10 to select between a CPR operation mode or a rescue breathing operation mode as shall be discussed in greater detail below. Mode change selector 30 may be a momentary switch, however other types of switches such as alternating action switches and tactile switches are also contemplated. In addition, CPR indicator 32 may indicate that CPR timer 10 has been placed in CPR operation mode, while the rescue breathing indicator 34 may indicate that CPR timer 10 has been placed in rescue breathing operation mode.

Patient selector 36 may allow the user to select between a child patient mode and an adult patient mode when the CPR timer 10 is in either the CPR operation mode or rescue breathing operation. In particular, child patient indicator 38 may indicate that CPR timer 10 is in the child patient mode and adult patient indicator 40 may indicate that CPR timer 10 is in the adult patient mode.

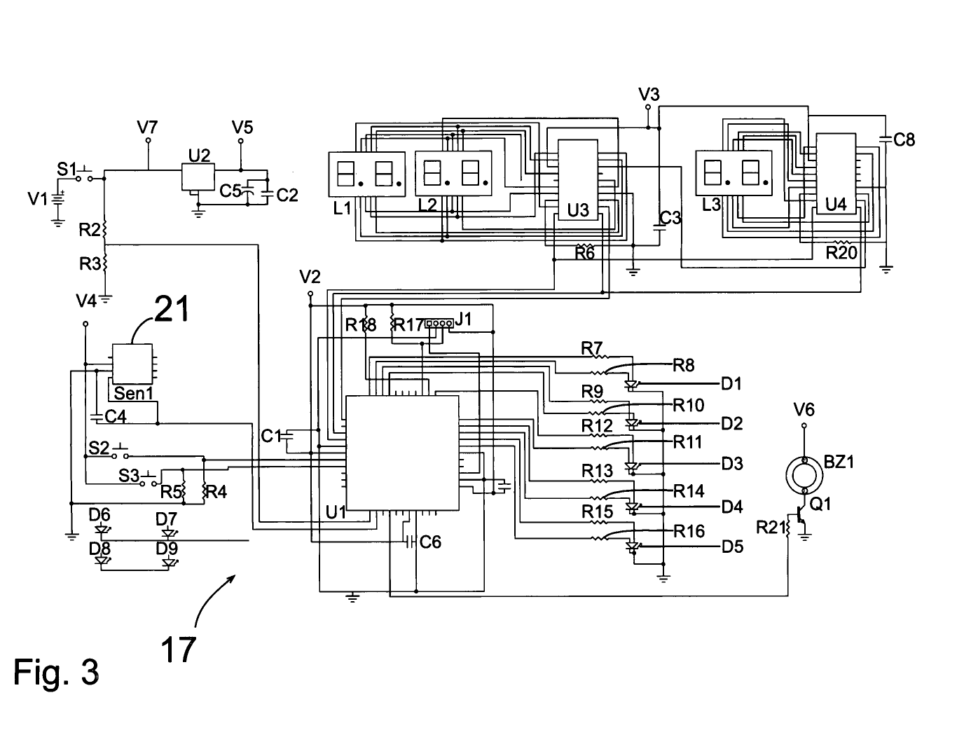

Referring to FIG. 3, an implementation of a schematic of CPR timer 10 is illustrated. In this implementation, a processor U1 may direct the various operations of CPR timer 10. Processor U1 may be a microcontroller or microprocessor, for example, a PIC16F872 processor manufactured by MICROCHIP or a MC9508AW16CF6E processor manufactured by Freescale, however other suitable processors are contemplated.

As shown, Input J1 may be electrically coupled to processor U1 through resistors R17 and R18 and capacitors C1, C6 and C7 and may enable programming of processor U1 by providing a data input. A crystal oscillator may be electrically coupled with processor U1. In this embodiment, Crystal oscillator acts as an internal clock to generate a timing signal to be used by processor U1. It should be appreciated that a crystal oscillator may not be required in implementations where processor U1 has an internal clock.

A series of light emitting diodes (“LEDs”) D1-D5 may be electrically coupled to processor U1 through resistors R7-R16 to provide CPR timer 10 with the necessary electronics for start indicator 20 and timing indicators 22, 24, 26, 28.

A non-visual indicator BZ1 may be electrically coupled to processor U1 through an amplifier Q1 and a resistor R21 to provide CPR timer 10 with a non-visual indicator. For example, amplifier Q1 may be a BC847AE6327 amplifier manufactured by FAIRCHILD SEMICONDUCTOR. In one embodiment, non-visual indicator BZ1 may be a noise generator such as a buzzer, while in another embodiment non-visual indicator BZ1 can be a vibratory component.

A selector S2 may be electrically coupled to processor U1. In addition, the mode change selector 30 may include selector S2 operatively associated with resistor R4. In addition, selector S3 may be electrically coupled to processor U1 and a resistor R5 to provide the electronics for patient mode selector 36. Diode pair D6 and D7 as well as diode pair D8 and D9 may be alternately illuminated in different order or in sequence to provide CPR indicator 32, rescue breathing indicator 34, patient selector 36, and child patient indicator 38, respectively.

A pressure sensor 21 may be operatively coupled to processor U1 in order to monitor pressure detected through flexible tubing 7 which is in fluid flow communication with valve assembly 3 through outlet port 15 in order to provide a means for the CPR timer 10 to determine whether the resuscitation bag 4 is being actuated and a sufficient breath is being delivered to the patient in order to initiate the pace setting operation. In one embodiment, pressure sensor 21 may continually monitor air pressure at a designated number of times per second, such as every 1/20 of a second, to determine if the monitored air pressure exceeds a predetermined threshold in order to initiate a pace setting operation. Pressure sensor 21 may be an integrated pressure sensor MPXV4006 manufactured by FREESCALE. Pressure sensor 21 may be capable of measuring air pressure in a range from 0-60 cm H₂0 above atmospheric pressure. A capacitor C4 may be electrically coupled to pressure sensor 21 and may be a 470 picofarad 25 v capacitor manufactured by SURGE.

Power may be provided to CPR timer 10 from a power source V1 when selector S1 is actuated. In one embodiment, power source V1 may be a 9 volt battery, however other suitable power sources are also contemplated. Selector S1 may be electrically coupled with resistors R2 and R3 to provide the electronics for ON/OFF switch 14 in order to either initiate or terminate operation of CPR timer 10.

Three seven-segment displays L1, L2 and L3 may be electrically coupled with drivers U3 and U4 to provide a numerical display for displaying the various values for inspiratory time indicator 16, BPM indicator 18 and airway pressure indicator 19. For example, drivers U3 and U4 may be an LED MC14489B driver made by MOTOROLLA.

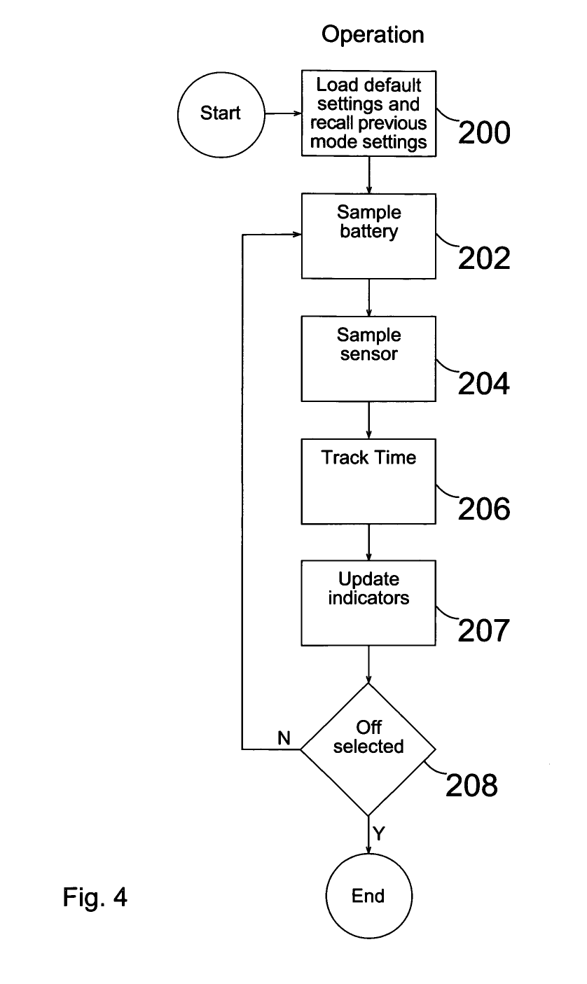

Referring to FIG. 4, an implementation of a method of operation for CPR timer 10 is illustrated. In this implementation, the default settings of CPR timer 10 may be loaded or the previous mode settings may be recalled at step 200. The default settings may be loaded when CPR timer 10 is turned on by actuation of ON/OFF switch 14.

In one embodiment, the mode settings of CPR timer 10 may be the settings for the operation mode or patient mode last used with CPR timer 10, however other embodiments such as having the predetermined mode settings set by the manufacturer or distributor are also contemplated. The default settings may be that non-visual indicator BZ1 is actuated rather than illuminating the timing indicators 22, 24, 26, 28.

At step 202, CPR timer 10 may sample a power source V1. An implementation of sampling a power source is described in greater detail below. CPR timer 10 may then sample pressure sensor SEN1 at step 204 in order to determine whether the resuscitation bag 4 has been actuated by the user. An implementation of sampling pressure sensor SEN1 is described in greater detail below.

At step 206, CPR timer 10 tracks the time once sufficient pressure has been detected. Thereafter, CPR timer 10 at step 207 may update the displays and indicators. For example, updating the settings may be checking for a change in operation mode or patient mode.

In one embodiment, when the CPR timer 10 is placed in the CPR operation mode the user provides life saving support by alternating between providing a breath to the patient by actuation of resuscitation bag 4 within a range of 1-1.4 seconds and then providing an appropriate number of chest compressions to the patient.

When in the rescue breathing operation mode, CPR timer 10 may be set to pace the user to provide 8 to 12 breaths a minute by having the user to maintain an inspiratory time of 1 to 1.4 seconds and an expiratory time of about 3.5 seconds when the patient is an adult, while a greater number of breaths per minute may be applied to a child by actuating the patient selector 36. In one embodiment, inspiratory time may indicate inhale time, while expiratory time may indicate the combination of exhale time and wait time.

In one embodiment, non-visual indicator BZ1 may be in ON or OFF mode by actuating mode change selector 30 for a predetermined period of time, such as two seconds, in order to provide an audio pace setting operation either alone or in combination with the visual pace setting operations discussed above. Non-visual indicator BZ1 may provide an audio indication, such as two beeps to indicate that non-visual indicator BZ1 has been turned ON or OFF.

CPR timer 10 at decision point 208 determines whether the OFF mode has been selected. In one embodiment, OFF mode may be selected when ON/OFF selector 14 is actuated by the user, however other embodiments such as holding down mode change selector 30 or patient selector 36 for a prescribed period of time are also contemplated.

If the OFF mode has not been selected by the user, CPR timer 10 returns to step 202. If OFF mode has been selected, an implementation of the foregoing method is complete.

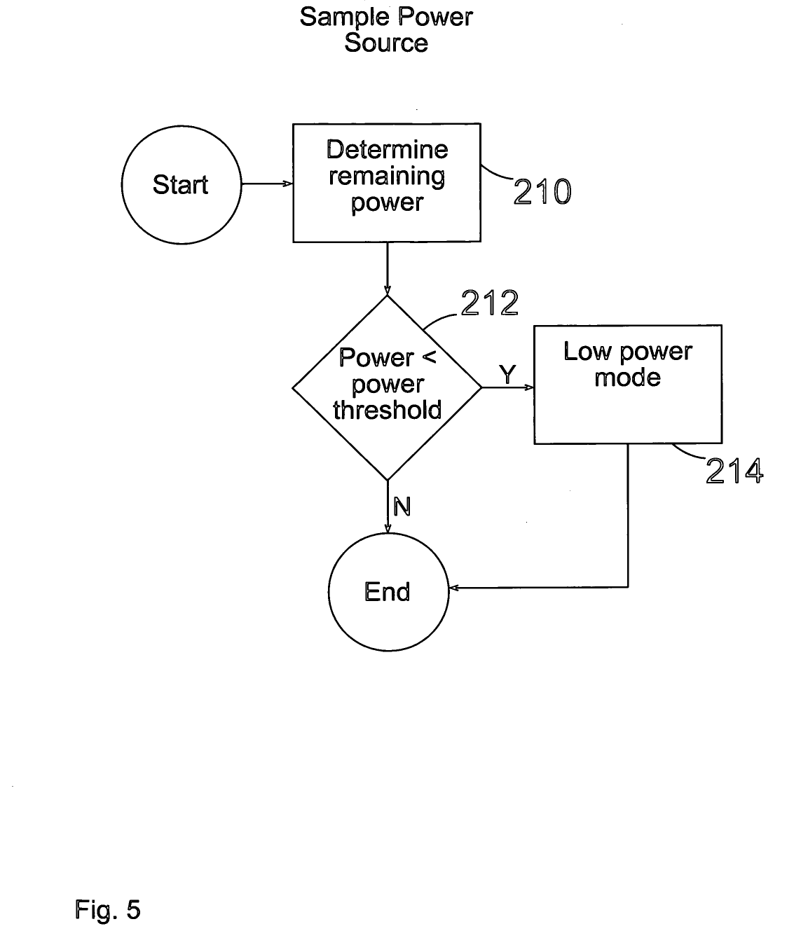

Referring to FIG. 5, an implementation of a method for sampling power source V1 is illustrated. In this implementation, CPR timer 10 at step 210 first determines the power remaining for operating CPR timer 12 at step 210. CPR timer 10 at decision point 212 may determine whether the remaining power of power source V1 is less than a predetermined power threshold. In one embodiment, the predetermined power threshold may be 6.7 volts, however other values may also be contemplated.

If the remaining power source V1 is less than the predetermined power threshold, CPR timer 10 may be placed in low power mode at step 214 for conserving power. In one embodiment, low power mode may not provide sufficient power to inspiratory time indicator 16 and BPM indicator 18, while continuing to power start indicator 20 and timing indicators 22, 24, 26, 28, however other low power arrangements are also contemplated.

In one embodiment, low power mode may be indicated by flashing one or more visual indicators, such as CPR indicator 32 and/or rescue breathing indicator 34. If the remaining power of power source V1 is not less than the predetermined power threshold, an implementation of the foregoing method is complete.

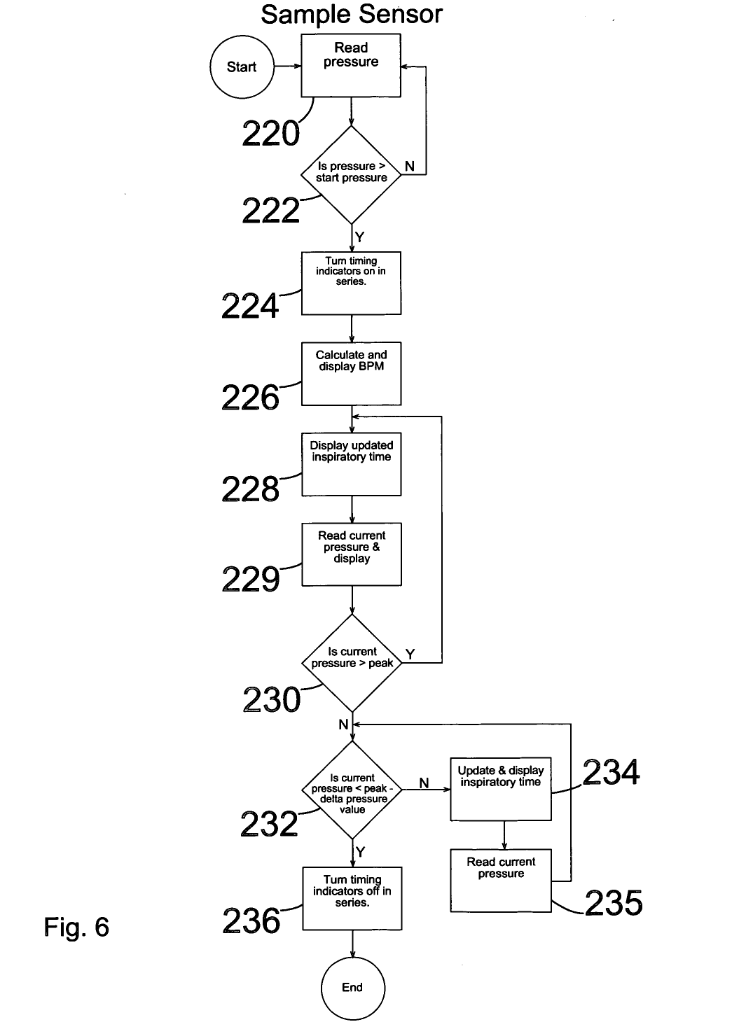

Referring to FIG. 6, an implementation of a method for sampling a sensor is illustrated. In this implementation, CPR timer 10 monitors pressure by pressure sensor 21 at step 220. At decision point 222, CPR timer 10 determines whether the current pressure is greater than the predetermined start pressure. If the current pressure is not greater than the predetermined start pressure, CPR timer 10 returns to step 220. If the current pressure is greater than the predetermined start pressure, CPR timer 12 may start tracking inspiratory time and proceed to step 224.

When the current pressure is greater than the predetermined start pressure CPR timer 10 may have detected the start of a breath to the patient by the bag mask resuscitor 2. In one embodiment, the predetermined start pressure may be 5 cm H₂O above atmospheric pressure, however other predetermined start pressures above and below 5 cm H₂0 atmospheric pressure are also contemplated.

CPR timer 10 may activate non-visual indicator BZ1 when the end of an Xₜᵢₘₑ occurs. The activation of non-visual indicator BZ1 may provide a “chirp” sound to indicate that the care giver should start a new breath and a “beep” sound to indicate that a new breath has started and detected by the CPR timer 10. In addition, non-visual indicator BZ1 may be activated for a predetermined period of time corresponding to the desired inspiratory time, such as one second.

At step 224, CPR timer 10 may sequentially activate timing indicators 22, 24, 26 28 in series to provide a visual pace setting indicator to guide the user for delivering a breath to the patient using resuscitation bag 4. In one embodiment, timing indicators 22, 24, 26 28 are sequentially illuminated so as to visually show elapsed time for pacing a properly timed breath to the patient such that all four timing indicators 22, 24, 26, 28 are all illuminated.

Alternatively, a properly timed breath may be delivered when indicators 20, 22, 24, and 26 are sequentially illuminated, but timing indicator 28 is not yet illuminated. In one embodiment, the timing indicators 22, 24, 26, 28 may be illuminated in an inspiratory color such as green, however other colors and/or color combinations are also contemplated.

At the end of desired inspiratory time, CPR timer 10 may calculate and display the breaths per minute being provided to the patient at step 226. In one embodiment, breaths per minute may be calculated by measuring the time between the start of the last two breaths and then dividing 60 by that number.

At step 228, CPR timer 10 may determine and display the updated inspiratory time on inspiratory time indicator 16. Thereafter, CPR timer 10 may read the current pressure detected by pressure sensor 21 at step 229.

CPR timer 10 at decision point 230 may determine whether the current detected pressure is greater than peak pressure previously detected by pressure sensor 21. If the current detected pressure is greater than peak pressure previously detected, CPR timer 10 returns to step 228 to display the updated inspiratory time. In one embodiment, CPR timer 10 may sample the current detected pressure twenty times a second, however other embodiments with different sampling rates are also contemplated. If the current detected pressure is not greater than peak pressure, CPR timer 10 proceeds to decision point 232.

CPR timer 10 may determine at decision point 232 whether the current detected pressure is less than the peak pressure minus a first predetermined delta pressure value, ΔP1. The first predetermined delta pressure value is a predetermined value for pressure that prevents inadvertent pressure surges in the bag mask resuscitator 2 from providing a false pressure reading. If the current detected pressure is not less than the peak pressure minus the first predetermined delta pressure value, CPR timer 10 updates and displays the inspiratory time at step 234 and then reads the current pressure at step 235. After the current pressure is read, the CPR timer 10 returns to decision point 232. If the current detected pressure is less than the peak pressure minus the first predetermined delta pressure value, CPR timer 10 proceeds to step 236.

CPR timer 10 at step 236 may sequentially deactivate timing indicators 22, 24, 26, 28 in reverse sequence to illustrate the expiratory phase when the patient is exhaling. In one embodiment, timing indicators 22, 24, 26, 28 may be deactivated sequentially in one second intervals when CPR timer 10 is in adult mode and in 4/10 of a second intervals when CPR timer 10 is in child mode, however other timing intervals are also contemplated. A unique expiratory color, for example yellow, may be activated for each timing indicator 22, 24, 26, 28 instead of deactivating timing indicators 22, 24, 26, 28 in reverse sequence. After completion of step 236, an implementation of the foregoing method is complete.

In one embodiment, after step 236 start indicator 20 may be illuminated and non-visual indicator BZ1 activated in order to provide a short audio notification to signify the start of a next breath by actuation of bag mask resuscitator 2.

A bad breath flag may be activated with the foregoing method to indicate that a particular breath applied to the patient was not within an acceptable breath range when detected by the pressure sensor 21. For example, an acceptable breath range may be 1.0 to 1.4 seconds of inspiratory time. CPR timer 10 may monitor whether the bad breath flag has been activated and may provide the user of CPR timer 10 with a bad breath alert. For example, the bad breath alert may be activated by changing start indicator 20 and/or one or more of timing indicators 22, 24, 26, 28 to a bad breath color for the duration of the breath and/or providing an audio notification. For example, bad breath color may be red, however the use of other colors and color combinations are also contemplated.

In one embodiment, a BPM flag may be used with the foregoing method to indicate that the breaths per minute is not within the predetermined acceptable BPM range. For example, the acceptable breaths per minute range may be 8-10 breaths per minute when the CPR timer 10 is in adult mode and 16-20 breaths per minute when the CPR timer 10 is in the child mode, however other acceptable BPM ranges are also contemplated.

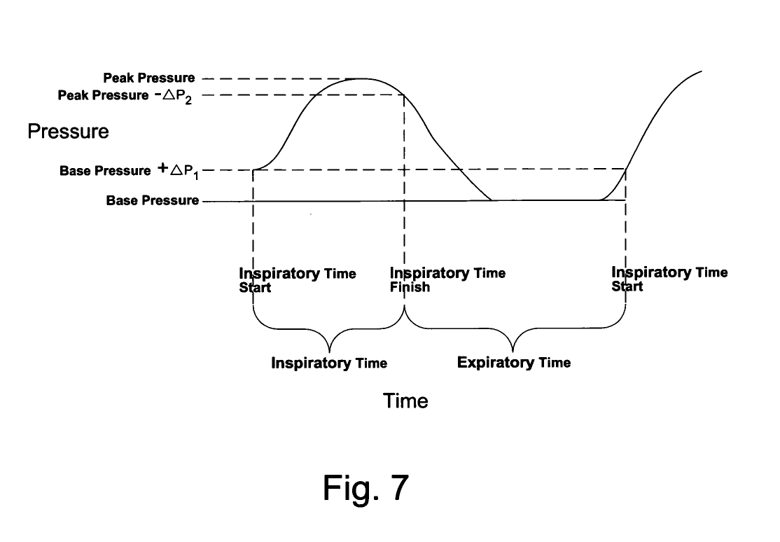

Referring to FIG. 7, a timing diagram illustrates a breath being provided to a patient using bag mask resuscitator 2 for the purpose of determining inspiratory and expiratory times for each breath given to a patient, while also determining the breaths per minute provided to the patient. In this implementation, the start of a breath may be shown to be provided by bag mask resuscitator 2 at a “Inspiratory Time Start” when pressure is set at Base Pressure+ΔP1, wherein Base Pressure is the lowest pressure achieved by the bag mask resuscitator 2 during operation and ΔP1 is the first predetermined delta pressure value. As the breath is begun to be provided to the patient by the actuation of the resuscitation bag 4, the current detected pressure will rise until the current detected pressure reaches the value of the Peak Pressure. The current detected pressure falls from the Peak Pressure as the a breath is continue to be provided to the patient until the current detected pressure reaches a value of Peak Pressure minus a second predetermined delta pressure value, ΔP2

In one embodiment, the first and second predetermined delta pressure values are 5 cm/H20, although other predetermined delta pressure values are contemplated. Once the current detected pressure reaches a value of Peak Pressure minus the second predetermined delta pressure value an “Inspiratory Time Finish” is established. Thereafter, the current detected pressure continues to fall until this pressure reaches the Base Pressure. Once current detected pressure falls to the Base Pressure, a delay may occur before the next breath is started by the actuation of the resuscitation bag 4. At the end of the delay, the current detected pressure may be seen to rise again from Base Pressure to Base Pressure+ΔP1 such that the next Peak Pressure may be determined.

As illustrated in FIG. 7, the time between the Inspiratory Time Start and Inspiratory Time Finish establishes the inspiratory time for that particular breath, while the time between the Inspiratory Time Finish and the next Inspiratory Time Start establishes the expiratory time for that particular breath. Accordingly, the inspiratory and expiratory times may be determined for each breath.

It should be understood from the foregoing that, while particular implementations have been illustrated and described, various modifications can be made thereto and are contemplated herein. It is also not intended that the invention be limited by the specific examples provided within the specification.

1. A cardiopulmonary resuscitation timer comprising:
a housing, said housing including a plurality of visual indicators arranged in an sequence, said plurality of visual indicators being adapted to be sequentially activated at a predetermined pace for providing breaths and/or chest compressions to a patient.

2. The cardiopulmonary resuscitation timer according to claim 1, wherein said plurality of visual indicator includes a start indicator and a plurality of timing indicators.

3. The cardiopulmonary resuscitation timer according to claim 2, wherein activation of said start indicator provides a visual cue to the user to begin providing breaths and/or chest compressions to a patient.

4. The cardiopulmonary resuscitation timer according to claim 2, wherein said plurality of timing indicators are sequentially activated in order to provide a visual pace setting device for pacing the frequency of breaths and/or chest compressions provided to a patient after the start indicator has been activated.

5. The cardiopulmonary resuscitation timer according to claim 1, wherein each of said plurality of visual indicators is an LED.

6. The cardiopulmonary resuscitation timer according to claim 1, wherein said predetermined pace includes providing a pace established for either an adult or a child.

7. The cardiopulmonary resuscitation timer according to claim 4, wherein said visual indicators are deactivated in reverse sequence during an expiratory time period.

8. The cardiopulmonary resuscitation timer according to claim 1, further including a pressure sensor for sensing air pressure indicative of a breath being provided to a patient.

9. The cardiopulmonary resuscitation timer according to claim 2, further including a pressure sensor for sensing air pressure indicative of a breath being provided to a patient, wherein said start indicator is activated when the air pressure sensed by said pressure indicator exceeds a predetermined threshold.

10. The cardiopulmonary resuscitation timer according to claim 2, wherein said plurality timing indicators are one color and said start indicator is a different color.

11. The cardiopulmonary resuscitation timer according to claim 2, further including an inspiratory time indicator for providing the amount of inspiratory time taken to provide one or more breaths to a patient.

12. The cardiopulmonary resuscitation timer according to claim 1, further including a breaths per minute indicator for indicating the number of breaths per minute provided to a patient.

13. The cardiopulmonary resuscitation timer according to claim 9, further including airway pressure indicator in operative association with said pressure indicator for indicating the presence of air pressure.

14. The cardiopulmonary resuscitation timer according to claim 2, wherein said timing indicators may be non-visual indicators.

15. The cardiopulmonary resuscitation timer according to claim 1, wherein said cardiopulmonary resuscitation timer is operable between a CPR operation mode and a rescue breathing operation mode.

16. The cardiopulmonary resuscitation timer according to claim 15, further including a mode change selector for changing the mode of operation between said CPR operation mode and a rescue breathing operation mode.

17. The cardiopulmonary resuscitation timer according to claim 16, further including a patient selector for providing different kinds of said predetermined pace when operating in either said CPR operation mode or a rescue breathing operation mode.

18. A bag mask resuscitator comprising:
a hollow, flexible resuscitation bag in fluid flow communication with a valve assembly through a hollow, flexible hose, said valve assembly being in operative engagement with a mask, said valve assembly including an adaptor defining an outlet port adapted to engage a hollow flexible tubing, and
a cardiopulmonary resuscitation timer defining an inlet port engaged to said tubing, said tubing in operative engagement with a pressure sensor for monitoring air pressure being applied by the bag mask resuscitator, said cardiopulmonary resuscitation timer further including a plurality of visual indicators for providing a predetermined pace for actuating said resuscitation bag.

19. The cardiopulmonary resuscitation timer according to claim 18, wherein said plurality of visual indicators are sequentially activated when said pressure sensor detects air pressure inside said tubing that exceeds a predetermined threshold.

20. A bag mask resuscitator comprising:
a hollow, flexible resuscitation bag in fluid flow communication with a valve assembly, said valve assembly being in operative engagement with a mask, said valve assembly including an adaptor defining an outlet port adapted to engage a hollow flexible tubing, and
a cardiopulmonary resuscitation timer defining an inlet port engaged to said tubing, said tubing in operative engagement with a pressure sensor for monitoring air pressure being applied by the bag mask resuscitator, said cardiopulmonary resuscitation timer further including a plurality of visual indicators for providing a predetermined pace for actuating said resuscitation bag.

21. The cardiopulmonary resuscitation timer according to claim 20, wherein said plurality of visual indicators are sequentially activated when said pressure sensor detects air pressure inside said tubing that exceeds a predetermined threshold.
